(19) United States
(12) Patent Application Publication
(10) Pub. No.: US 2024/0033483 A1
(43) Pub. Date: Feb. 1, 2024

(54) CATHETER WITH PULL WIRE BAND ELECTRODE

(71) Applicant: Medtronic, Inc., Minneapolis, MN (US)

(72) Inventor: Kevin A. Knutsen, Ramsey, MN (US)

(21) Appl. No.: 18/358,705

(22) Filed: Jul. 25, 2023

Related U.S. Application Data
(60) Provisional application No. 63/369,609, filed on Jul. 27, 2022.

(51) Int. Cl. A61M 25/01; A61M 25/00

(52) U.S. Cl. CPC A61M 25/0147; A61M 25/0136; A61M 25/0045

(57) ABSTRACT

A catheter includes a control handle and a catheter shaft coupled to the control handle and extending from the control handle. The catheter shaft has a proximal end coupled to the control handle and a distal end located opposite the proximal end. The catheter also includes a pull wire band coupled to the catheter shaft. At least a portion of the pull wire band is exposed to an environment outside of the catheter shaft. The catheter also includes a pull wire extending from the control handle and through the catheter shaft to the pull wire band.

### CROSS-REFERENCE TO RELATED APPLICATIONS

This application claims priority to U.S. Provisional Application No. 63/369,609, filed Jul. 27, 2022, the entire contents of which are hereby incorporated by reference.

### BACKGROUND

Catheters, including delivery catheters, commonly include the use of one or more pull wires that are used to deflect and steer a distal end of the catheter during a procedure. By manipulating an actuating mechanism at a control handle of the catheter, the pull wire or wires are tensioned, causing the distal end of the delivery catheter to deflect.

During some procedures, such as treatment of atrial fibrillation, the distal end of a delivery catheter is first moved and steered into the left atrium of the heart. A separate diagnostic catheter (e.g., electrophysiology catheter) is then inserted through the delivery catheter. The diagnostic catheter includes one or more electrodes that detect and measure electrical signals coming from the pulmonary veins. Those signals are then sent back through the diagnostic catheter, and the signals are recorded and mapped. The diagnostic catheter is then removed from the delivery catheter, and an ablation catheter (e.g., cryoballoon catheter) is inserted through the delivery catheter. The ablation catheter is used to deliver ablation energy (e.g., cold energy via a refrigerant), creating scar tissue at the opening of the pulmonary vein. This scar tissue is intended to stop the transmission of electrical signals that cause atrial fibrillation. Once ablation is complete, the ablation catheter is removed. The diagnostic catheter is then re-inserted through the delivery catheter, and measurements of the electrical signals are again taken to determine whether the ablation was successful.

### SUMMARY

One example provides a catheter that includes a control handle and a catheter shaft coupled to the control handle. The catheter shaft extends from the control handle. The catheter shaft also has a proximal end coupled to the control handle and a distal end located opposite the proximal end. The catheter also includes a pull wire band coupled to the catheter shaft. At least a portion of the pull wire band is exposed to an environment outside of the catheter shaft. The catheter also includes a pull wire extending from the control handle and through the catheter shaft to the pull wire band.

Other aspects, examples, instances, and various embodiments will become apparent by consideration of the detailed description and accompanying drawings.

### BRIEF DESCRIPTION OF THE DRAWINGS

FIG. 1 is a perspective view of a catheter according to one example.

FIG. 2 is an enlarged view of a distal end of the catheter, illustrating an exposed region of a pull wire band.

FIG. 3 is an enlarged view of the distal end of the catheter, illustrating a non-exposed region of the pull wire band.

FIG. 4 is a schematic, cross-sectional view of the distal end of the catheter according to one example, illustrating pull wires coupled to the pull wire band.

FIG. 5 is a schematic, cross-sectional view of the distal end of the catheter according to another example, illustrating an additional layer of the pull wire band.

FIG. 6 is a partial cut-away view of a control handle of the catheter, illustrating an actuating mechanism for the pull wires.

FIG. 7 is a side view of the control handle of the catheter, illustrating an electrode wire.

FIG. 8 is a side view of a portion of an interior of the control handle, illustrating a connection of the electrode wire to one of the pull wires.

### DETAILED DESCRIPTION

Before aspects, examples, instances, and any embodiments are explained in detail, it is to be understood that embodiments are not limited in their application to the details of construction and the arrangement of components set forth in the following description or illustrated in the following drawings. Other aspects, examples, instances, and embodiments are possible and aspects, examples, instances, and embodiments described and illustrated are capable of being practiced or of being carried out in various ways.

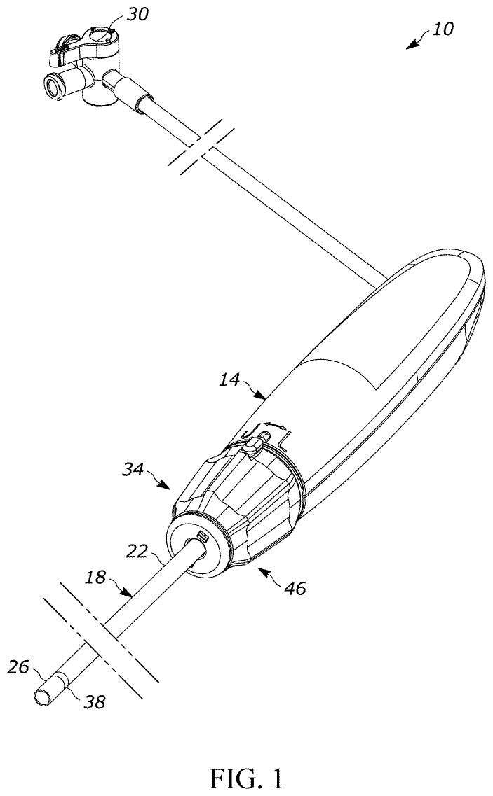

FIGS. 1-8 illustrate an example of a catheter 10. In the example illustrated, the catheter 10 is a delivery catheter, designed and configured to be used for example during treatment of atrial fibrillation or during other medical procedures. The catheter 10 includes a control handle 14 and a catheter shaft 18 (e.g., flexible polymer shaft) coupled to and extending from the control handle 14. The catheter shaft 18 has a proximal end 22 coupled to the control handle 14 and a distal end 26. The distal end 26 is located opposite the proximal end 22, such that along a longitudinal axis defined by the catheter 10 the proximal end 22 is at one end of catheter 10 and the distal end 26 is at the other end. In the illustrated example, the catheter 10 also includes a stopcock 30 that allows for flushing of the catheter 10. The catheter 10 also includes a valve (not shown) at a proximal end of the control handle 14 that allows for introduction of other instruments and/or materials (e.g., ablation catheters) through the catheter 10.

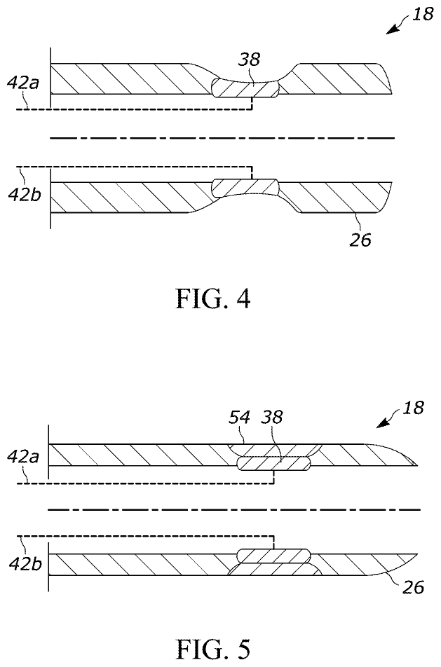

As best seen by reference to FIGS. 1-5, the catheter 10 also includes a steering system 34 to deflect and steer the distal end 26 of the catheter shaft 18. The steering system 34 includes a pull wire band 38 coupled to the catheter shaft 18. In one aspect, the pull wire band 38 is located at the distal end 26 of the catheter shaft 18. The steering system 34 also includes at least one pull wire 42a, 42b (illustrated schematically in FIGS. 4 and 5) extending from the control handle 14 and through the catheter shaft 18 to the pull wire band 38, and an actuating mechanism 46 (FIG. 1) coupled to the pull wire (e.g., wires 42a, 42b) to tension the pull wire (e.g., wires 42a, 42b) and deflect the distal end 26 of the catheter shaft 18. During operation of the steering system 34, when the actuating mechanism 46 is activated (e.g., twisted), the distal end 26 of the catheter shaft 18 is deflected. For example, deflecting the distal end 26 may include a distal tip curving or bending backwards towards a portion of the catheter shaft 18 that is proximal to the distal end 26. The distal end 26 may deflect at a single point, such that the distal end 26 defines an angle related to that portion of the catheter shaft 18 that is proximal to the distal end 26. For example, the distal end 26 may be configured to define an angle relative to a previously undeflected position, such that relative to the previously undeflected position the distal end 26 deflects an angle, e.g., up to 90 degrees, up to 135 degrees, or other values and ranges of values. As used herein, deflection may serve the purpose of moving an electrode or electrodes (e.g., the pull wire band 38) at the distal end 26 of the catheter shaft 18 to a position adjacent a targeted tissue site so that the electrode(s) may provide therapy to the tissue and/or diagnose the tissue. With reference to FIG. 4, in the illustrated example the steering system 34 includes a first pull wire 42a and a second pull wire 42b coupled (e.g., fixed) to opposite sides of the pull wire band 38. Each of the first and second pull wires 42a, 42b extends from the pull wire band 38 to the control handle 14. In some examples, the pull wires 42a, 42b lay flat along the pull wire band 38 (e.g., along an exposed portion of the pull wire band 38, and/or between layers of reflowed polyether block amide or other material). For example, portions of the pull wires 42a, 42b may be fixed to the pull wire band 38, and/or portions of the pull wires 42a, 42b may have fixed shapes that cause the pull wires 42, 42b to contact the pull wire band 38. Other examples include different arrangements, orientations, and positions of the pull wires 42a, 42b than that illustrated and described.

With continued reference to FIGS. 1-5, at least one region of the pull wire band 38 (e.g., a portion of an outer surface of the pull wire band 38) is exposed to an environment outside of the catheter shaft 18. Exposing the pull wire band 38 allows the pull wire band 38 itself to serve as an electrode while still being used as part of the steering system. During use, the exposed portion of pull wire band 38 may come into contact with tissue of a human body (e.g., within a pulmonary vein), and is not insulated from electrical signals within the human body (e.g., signals associated with a heartbeat at the pulmonary vein). The exposed region or regions of the pull wire band 38 are thus able to receive electrical signals, allowing the catheter 10 to function as a diagnostic catheter in addition to, or alternatively to, functioning as a delivery catheter.

According to one aspect, the pull wire band 38 is generally circular in shape, although in other aspects and examples the pull wire band 38 has different shapes (e.g., elliptical, etc.). In some examples, the pull wire band 38 is made at least partially of one or more of platinum, stainless steel, or gold, although other examples include other materials and combinations of materials (e.g., electrically conductive materials). Overall, the number and size of the exposed region or regions of the pull wire band 38 are selected such that enough of the pull wire band 38 is exposed to the environment outside of the catheter shaft 18 to receive electrical signals (i.e., to enable the pull wire band 38 to serve as an electrode), but enough of the pull wire band 38 is still embedded or otherwise fixed to the catheter shaft 18 so that the pull wire band 38 remains part of the steering system 34 to deflect and steer the distal end 26 of the catheter shaft 18. In some instances, approximately 75% of the pull wire band 38 (e.g., approximately 75% of an outer surface of the pull wire band 38) is exposed to the outside environment, whereas approximately 25% or less of the pull wire band 38 (e.g., approximately 25% of the outer surface of the pull wire band 38) remains embedded or otherwise unexposed. In other instances, between 70% to 80% of the pull wire band 38 is exposed, or between 60% and 90% of the pull wire band 38 is exposed. Other aspects include different values and ranges of values.

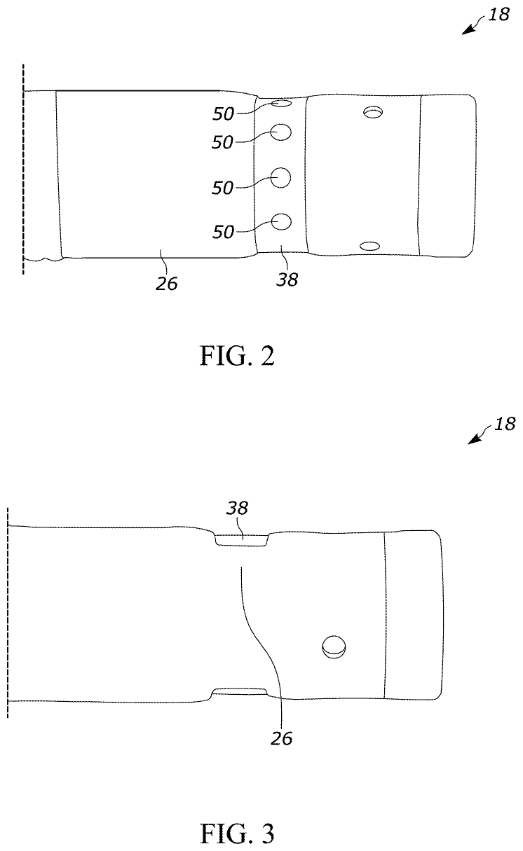

With reference to FIGS. 2-4, in some examples the exposed region or regions of the pull wire band 38 are recessed (e.g., radially recessed) relative to an outer surface of the surrounding material of the catheter shaft 18. For example, in some examples, during production of the catheter 10 the pull wire band 38 is first entirely embedded within the catheter shaft 18 (e.g., in a co-molding operation). A portion of the catheter shaft 18 is then removed (e.g., mechanically stripped away), thereby exposing the underlying region (or regions) of pull wire band 38. With reference to FIG. 2, in some examples, small openings (e.g., circular openings) 50 in the pull wire band 38 are exposed. These openings 50 are spaced circumferentially apart from one another, and are used for example during the production process, to allow the material of the catheter shaft 18 to flow into the openings 50 and thereby secure the pull wire band 38 to the catheter shaft 18.

With reference to FIG. 5, in other examples the pull wire band 38 includes an additional layer or layers 54 of material that are positioned on top of (e.g., added onto or integrally formed in a single piece with) the underlying portions of the pull wire band 38, such that an outer surface of the exposed region (or regions) of the overall pull wire band 38 are substantially flush with an outer surface of the catheter shaft 18. The additional layer or layers 54 may be added, for example, after a step of removing a portion of the catheter shaft 18 to expose the pull wire band 38. In some examples, the additional layer or layers 54 extend between 10%-40% around the catheter shaft 18 (e.g., around a circumference of the catheter shaft 18), or between 20% and 30% around the catheter shaft 18, or other values and ranges of values. The additional layer or layers 54 may be formed of the same material as a remainder of the pull wire band 38. For example, the additional layer or layers 54 may be formed at least in part of one or more of platinum, stainless steel, or gold, or other electrically conductive materials.

In some instances, the pull wire band 38 (with or without the additional layer or layers 54) is formed together (e.g., co-molded or otherwise integrally formed together as a single piece) with the catheter shaft 18, such that the exposed region or regions of the pull wire band 38 are already exposed along the outside of the catheter shaft 18 upon initial formation of the catheter 10.

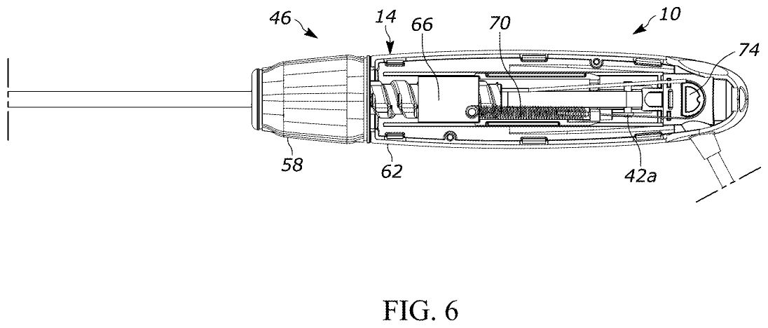

With reference to FIG. 6, in the illustrated example the actuating mechanism 46 includes a rotatable control knob 58 located at a distal end of the control handle 14, a drive screw 62 coupled to and rotationally fixed to the control knob 58, a slider 66 coupled to the drive screw 62, and a biasing element (e.g., spring) 70 coupled to the slider 66. The first pull wire 42a extends from the pull wire band 38 and through the catheter shaft 18, and wraps around a D-shaped guide 74 located within the control handle 14. The first pull wire 42a extends through the biasing element 70 and is coupled (e.g., fixed) to the slider 66.

If the control knob 58 is rotated in a first direction, the drive screw 62 is also rotated, which imparts a linear translation to the slider 66. As the slider 66 translates (e.g., to the left in FIG. 6), the slider 66 pulls on the first pull wire 42a, tensioning the first pull wire 42a and thereby causing the pull wire band 38 (and the connected distal end 26 of the catheter shaft 18) to deflect and flex (e.g., up to 90 degrees, up to 135 degrees, or other values and ranges of values). As the slider 66 translates, the biasing element 70 is also tensioned, such that the slider 66 is biased back (i.e., to the right in FIG. 6) when the control knob 58 is released.

If the control knob 58 is rotated in a second direction, opposite the first direction, the second pull wire 42b may be tensioned, causing the distal end 26 of the catheter shaft 18 to deflect and flex (e.g., up to 90 degrees, up to 135 degrees, or other values and ranges of values) in a different (e.g., opposite) direction.

Other aspects include different actuating mechanisms 46 than those illustrated, and also different numbers and arrangements of pull wires 42a, 42b. For example, in some instances the actuating mechanism 46 includes a control knob 58 (or other control structure such as a button, slide, etc.) at a different location than that illustrated, or includes more than one control knob 58, or includes a control structure that is translated (e.g., a thumb slide) or depressed (e.g., a button) rather than rotated to cause tensioning in a pull wire. Additionally, in some instances the actuating mechanism 46 does not include one or more of the drive screw 62, slider 66, biasing element 70, or D-shaped guide 74, or includes a different arrangement of a drive screw 62, slider 66, biasing element 70, and/or D-shaped guide 74 than that illustrated. In some instances, only a single pull wire 42a, 42b is used, for example to cause the distal end 26 of the catheter shaft 18 to deflect and flex in a single direction, or includes more than two pull wires that can be used to deflect and steer the distal end 26 of the catheter 10 in various directions.

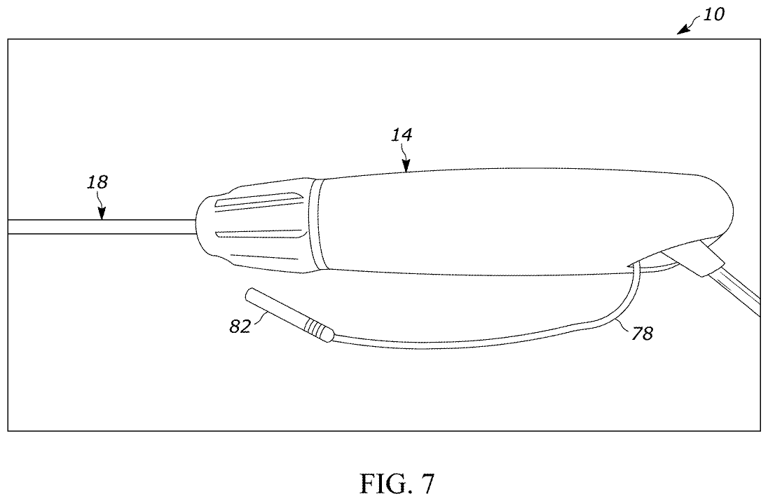

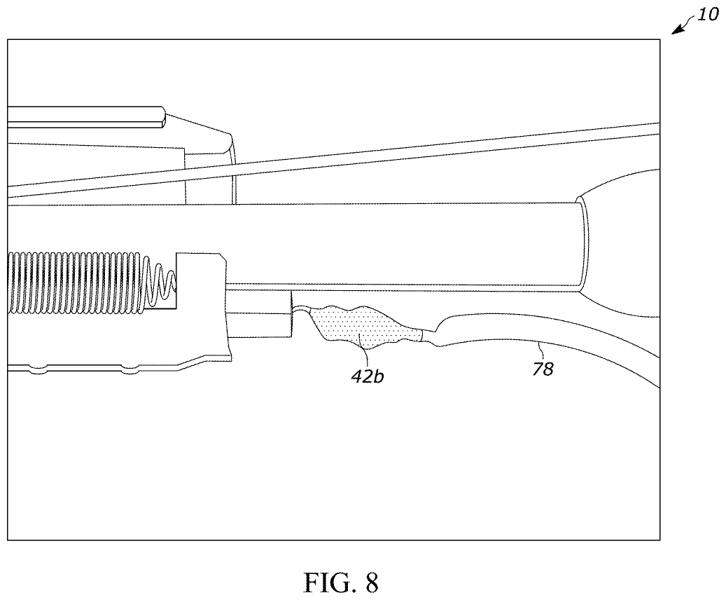

With reference to FIGS. 7 and 8, and as described above, the pull wire band 38 functions both as part of the steering system 34 for the catheter 10 and also as an electrode (e.g., for diagnostic purposes). For this reason, the catheter 10 also includes an electrode wire 78 coupled to at least one of the pull wires 42a, 42b, such that the electrical signals received by the pull wire band 38 may be transferred through one or more of the pull wires 42a, 42b and out of the catheter 10 (e.g., to then be recorded and mapped for diagnostic purposes). In one example, the electrode wire 78 is coupled (e.g., soldered) at one end to the second pull wire 42b within the control handle 14, and includes a plug 82 at an opposite end. In other instances, the electrode wire 78 is coupled (e.g., soldered) at one end to the first pull wire 42a, or to both the first pull wire 42a and the second pull wire 42b, or to a different pull wire other than that illustrated.

During use, electrical signals are received at the exposed region of the pull wire band 38, and are sent through the first and/or second pull wires 42a, 42b and into the electrode wire 78. The pull wire or wires 42a, 42b thereby serve not only to facilitate steering of the distal end 26 of the catheter 10, but also to facilitate transmission of signals from the electrode pull wire band 38. In some examples, the electrode wire 78 is coupled (e.g., plugged into) electrical equipment (e.g., a controller, processor, display, or other equipment) that is used to record and/or map the signals.

Other aspects and examples include different arrangements than that illustrated. For example, in one instance the electrode wire 78 is coupled to the pull wire 42a and/or the pull wire 42b at a location that is outside the control handle 14, or otherwise in a different location than that illustrated. Additionally, some examples include more than one electrode wire 78 (e.g., separate electrode wires 78 coupled to different pull wires), and/or do not include a plug 82 at one end of the electrode wire 78.

The examples may be further described by reference to the following numbered paragraphs:

1. A catheter comprising:
a control handle;
a catheter shaft coupled to the control handle and extending from the control handle, the catheter shaft having a proximal end coupled to the control handle and a distal end located opposite the proximal end;
a pull wire band coupled to the catheter shaft, wherein at least a portion of the pull wire band is exposed to an environment outside of the catheter shaft; and
a pull wire extending from the control handle and through the catheter shaft to the pull wire band.
   2. The catheter of paragraph 1, wherein between 60% to 90% of an outer surface of the pull wire band is exposed to the environment outside of the catheter shaft.
   3. The catheter of paragraph 2, wherein approximately 75% of the outer surface of the pull wire band is exposed to the environment outside of the catheter shaft.
   4. The catheter of paragraph 1, wherein the portion of the pull wire band that is exposed to the environment outside of the catheter shaft is recessed relative to an outer surface of the catheter shaft.
   5. The catheter of paragraph 1, wherein the pull wire band includes an additional layer having an outer surface that is substantially flush with an outer surface of the catheter shaft.
   6. The catheter of paragraph 5, wherein the additional layer is formed of the same material as a remainder of the pull wire band.
   7. The catheter of paragraph 6, wherein the additional layer is formed at least in part from an electrically conductive material.
   8. The catheter of paragraph 5, wherein the additional layer extends between 20% to 30% around a circumference of the catheter shaft.
   9. The catheter of paragraph 1, wherein the pull wire band includes openings spaced circumferentially apart.
   10. The catheter of paragraph 1, wherein the pull wire band is coupled to the distal end of the catheter shaft.
   11. The catheter of paragraph 1, wherein the pull wire band is an electrode.
   12. The catheter of paragraph 11, wherein the pull wire band is part of a steering system for deflecting and steering the distal end of the catheter.
   13. The catheter of paragraph 12, wherein the steering system includes the pull wire band, the pull wire, and an actuating mechanism at the control handle.
   14. The catheter of paragraph 13, wherein the actuating mechanism includes a control knob, wherein the control knob is coupled to the pull wire such that when the control knob is rotated, the pull wire is tensioned, and the distal end of the catheter flexes.
   15. The catheter of paragraph 1, further comprising an electrode wire coupled to the pull wire.
   16. The catheter of paragraph 15, wherein the electrode wire is coupled to the pull wire within the control handle.
   17. The catheter of paragraph 1, wherein the pull wire band is formed at least in part from an electrically conductive material.
   18. The catheter of paragraph 1, wherein the catheter is a delivery catheter.
   19. The catheter of paragraph 18, wherein the delivery catheter includes a stopcock.
   20. The catheter of paragraph 1, wherein the catheter shaft is a flexible polymer shaft.

Although various aspects and examples have been described in detail with reference to certain examples illustrated in the drawings, variations and modifications exist within the scope and spirit of one or more independent aspects described and illustrated.

What is claimed is:

1. A catheter comprising:
a control handle;
a catheter shaft coupled to the control handle and extending from the control handle, the catheter shaft having a proximal end coupled to the control handle and a distal end located opposite the proximal end;
a pull wire band coupled to the catheter shaft, wherein at least a portion of the pull wire band is exposed to an environment outside of the catheter shaft; and
a pull wire extending from the control handle and through the catheter shaft to the pull wire band.

2. The catheter of claim 1, wherein between 60% to 90% of an outer surface of the pull wire band is exposed to the environment outside of the catheter shaft.

3. The catheter of claim 2, wherein approximately 75% of the outer surface of the pull wire band is exposed to the environment outside of the catheter shaft.

4. The catheter of claim 1, wherein the portion of the pull wire band that is exposed to the environment outside of the catheter shaft is recessed relative to an outer surface of the catheter shaft.

5. The catheter of claim 1, wherein the pull wire band includes an additional layer having an outer surface that is substantially flush with an outer surface of the catheter shaft.

6. The catheter of claim 5, wherein the additional layer is formed of the same material as a remainder of the pull wire band.

7. The catheter of claim 6, wherein the additional layer is formed at least in part from an electrically conductive material.

8. The catheter of claim 5, wherein the additional layer extends between 20% to 30% around a circumference of the catheter shaft.

9. The catheter of claim 1, wherein the pull wire band includes openings spaced circumferentially apart.

10. The catheter of claim 1, wherein the pull wire band is coupled to the distal end of the catheter shaft.

11. The catheter of claim 1, wherein the pull wire band is an electrode.

12. The catheter of claim 11, wherein the pull wire band is part of a steering system for deflecting and steering the distal end of the catheter.

13. The catheter of claim 12, wherein the steering system includes the pull wire band, the pull wire, and an actuating mechanism at the control handle.

14. The catheter of claim 13, wherein the actuating mechanism includes a control knob, wherein the control knob is coupled to the pull wire such that when the control knob is rotated, the pull wire is tensioned, and the distal end of the catheter flexes.

15. The catheter of claim 1, further comprising an electrode wire coupled to the pull wire.

16. The catheter of claim 15, wherein the electrode wire is coupled to the pull wire within the control handle.

17. The catheter of claim 1, wherein the pull wire band is formed at least in part from an electrically conductive material.

18. The catheter of claim 1, wherein the catheter is a delivery catheter.

19. The catheter of claim 18, wherein the delivery catheter includes a stopcock.

20. The catheter of claim 1, wherein the catheter shaft is a flexible polymer shaft.
